(19) United States
(12) Patent Application Publication
(10) Pub. No.: US 2010/0137898 A1
(43) Pub. Date: Jun. 3, 2010

(54) Vaso-occlusive devices with attachment assemblies for stretch-resistant members

(75) Inventor: Clifford Teoh, Los Altos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/589,717

(22) Filed: Oct. 27, 2009

Related U.S. Application Data
(60) Provisional application No. 61/200,685, filed on Dec. 2, 2008.

(51) Int. Cl. A61M 29/00; A61F 2/00

(52) U.S. Cl. 606/195; 623/1.11

(57) ABSTRACT

Disclosed herein are vaso-occlusive devices for forming occluding the vasculature of a patient. More particularly, disclosed herein are vaso-occlusive devices comprising an implantable device, an attachment assembly and a stretch-resistant member secured to the attachment assembly and to the implantable device. Also disclosed are methods of making and using these devices.

### CROSS-REFERENCE TO RELATED APPLICATIONS

This application claims the benefit of U.S. provisional patent application No. 61/200,685 filed Dec. 2, 2008, the disclosure of which is incorporated by reference in its entirety for all purposes.

### FIELD OF THE INVENTION

Devices and methods for repair of aneurysms are described. In particular, attachment assemblies for vaso-occlusive devices are described.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.).

Coil devices including polymer coatings or attached polymeric filaments have also been described. See, e.g., U.S. Pat. Nos. 5,226,911; 5,935,145; 6,033,423; 6,280,457; 6,287,318; and 6,299,627. For instance, U.S. Pat. No. 6,280,457 describes wire vaso-occlusive coils having single or multi-filament polymer coatings. U.S. Pat. Nos. 6,287,318 and 5,935,145 describe metallic vaso-occlusive devices having a braided polymeric component attached thereto. U.S. Pat. No. 5,382,259 describes braid structures covering a primary coil structure.

In addition, coil designs including stretch-resistant members comprising thermoplastic polymeric fibers that run through the lumen of the helical vaso-occlusive coil and are secured to the coil by heat treatment have also been described. See, e.g., U.S. Pat. Nos. 5,582,619; 5,833,705; 5,853,418; 6,004,338; 6,013,084; 6,179,857; 6,165,178; and 6,193,728. Stretch-resistant coils are also described in U.S. Patent Publication Nos. 2007/0239193 and 2006/0271086.

Coils are typically deployed into the site of choice by physical means and/or by application of electrical and/or thermal energy. For example, U.S. Pat. Nos. 6,620,152; 6,425,893; 5,976,131 5,354,295; and 5,122,136, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint. However, in some vaso-occlusive devices thermal or electrical detachment can compromise the stretch-resistant members (e.g., by degrading the anchor point), which renders the coils vulnerable to stretching after deployment.

Thus, there remains a need for attachment assemblies for vaso-occlusive devices to make stretch-resistant vaso-occlusive devices that maintain their stretch-resistant properties with respect to the coil after deployment or with respect to the delivery device. There also remains a need for as methods of making and using such devices.

### SUMMARY

Thus, this invention includes novel occlusive compositions as well as methods of using and making these compositions.

In one aspect, described herein is a vaso-occlusive device comprising an implantable device having a proximal end and a distal end; an attachment assembly having proximal and distal ends, the attachment assembly disposed at or near the proximal end of the implantable device; and at least one stretch-resistant member secured to the implantable device and the attachment assembly. In certain embodiments, the attachment assembly comprises at least one lumen therein and wherein at least one stretch-resistant member (e.g., at least one filament) extends through the lumen of the attachment assembly. In any of the devices described herein, the stretch-resistant member can be secured to the proximal and/or distal end of the attachment assembly. The stretch-resistant member can be made of any material (e.g., a polymer such as a suture material) and can be secured to the attachment assembly by knotting, adhesive or any other bonding mechanism.

In certain aspects, the attachment assembly comprises a metal, for example platinum. In other embodiments, the implantable device defines a lumen and the attachment assembly extends at least partially into the lumen of the implantable device. In any of the devices described herein, the attachment assembly may be secured to the implantable device.

In certain embodiments, the implantable device comprises a wire formed into a helically wound primary shape. The implantable device may also have a secondary shape (e.g., cloverleaf shaped, helically-shaped, figure-8 shaped, flower-shaped, vortex-shaped, ovoid, randomly shaped, and substantially spherical shapes) that self-forms upon deployment.

In yet another aspect, provided herein is an assembly comprising any of the vaso-occlusive devices as described herein and a delivery device (e.g., pusher wire, delivery coil, etc.), wherein the vaso-occlusive device is secured to the delivery device prior to deployment.

In any of the assemblies or devices described herein, the implantable device can comprise a metal, for example, platinum, rhodium, gold, tungsten and/or alloys thereof. In certain embodiments, the implantable device comprises a nickel-titanium alloy.

Any of the assemblies or devices described herein may further comprise one or more additional materials, for example, at least one bioactive material.

Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof.

In another aspect, a method of occluding a body cavity is described, the method comprising introducing any of the devices as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is an overview depicting an exemplary attachment assembly as described herein. The embodiment shown is a cylindrical plug shape with 2 lumens therein.

FIG. 2 is a side view showing the exemplary attachment assembly of FIG. 1 with a suture threaded through the lumens of the attachment assembly and tethered by knotting at each end of the attachment assembly. The suture may also act as a stretch-resistant member when combined with a vaso-occlusive coil.

FIG. 3 is a side view depicting an exemplary assembly as described herein and shows the attachment assembly of FIG. 2, vaso-occlusive coil implant 30, delivery loop 70 and detachment junction 60 formed by attaching the attachment assembly to the delivery loop via the proximal suture.

FIG. 4 is a side view of the assembly of FIG. 3 after detachment (e.g., severing) of the device and attachment assembly from the delivery loop.

FIG. 5 is a side view depicting another exemplary assembly as described herein in which the attachment assembly is positioned within the lumen of the implantable coil.

FIG. 6 is a side view of the assembly of FIG. 5 after detachment (e.g., severing) of the device and attachment assembly structure from the delivery loop.

### DETAILED DESCRIPTION

Stretch-resistant occlusive (e.g., embolic) devices and assemblies are described. The compositions described herein find use in vascular and neurovascular indications and are particularly useful in treating aneurysms, for example small-diameter, curved or otherwise difficult to access vasculature, for example aneurysms, such as cerebral aneurysms. Methods of making and using these vaso-occlusive devices are also aspects of this invention.

Unlike certain previously described stretch-resistant vaso-occlusive coils, the devices described herein maintain their stretch-resistant function after deployment, for example, when degradation of the deployment junction also releases, degrades or severs one or more stretch-resistant member(s) and/or the attachment assembly(ies) for the stretch-resistant members.

Advantages of the present invention include, but are not limited to, (i) the provision of stretch-resistant vaso-occlusive devices with high tensile strength; (ii) the provision of stretch-resistant devices that result in structures having more uniform dimensions (e.g., in terms of the outer diameter remaining more consistent along its entire length); (iv) the provision of occlusive devices that can be retrieved and/or repositioned after deployment; and (v) cost-effective production of these devices.

All publications, patents and patent applications cited herein, whether above or below, are hereby incorporated by reference in their entirety.

It must be noted that, as used in this specification and the appended claims, the singular forms “a”, “an”, and “the” include plural references unless the content clearly dictates otherwise. Thus, for example, reference to a device comprising “a stretch-resistant member” includes devices comprising of two or more stretch-resistant members.

The devices described herein comprise an attachment assembly. Preferably, the attachment assembly is situated between the detachment zone and the proximal end of the implantable device (coil) and is configured such that the stretch-resistant member of the implantable device continues to prevent significant stretching of the device after deployment.

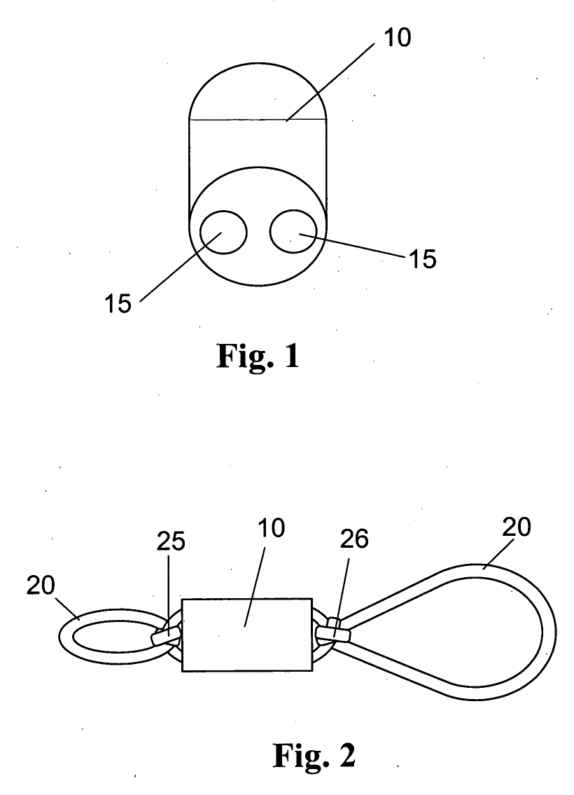

The attachment assembly may take a variety of forms including disk shapes, cylinders, ball (sphere) shapes, ovoid shapes, half-spheres, half-ovals, cones, etc. The attachment assembly preferably includes at least one lumen through which one or more filaments (e.g., one or more of the stretch resistant members) extend. Alternatively, the attachment assembly may include one or more channels on its exterior surface around which the stretch-resistant members may be wound. FIG. 1 shows an exemplary attachment assembly 10 comprising a cylindrical plug with multiple lumens 15 therein.

The attachment assembly may be made of any metal, polymer or other implantable material including, but not limited to, the metals, polymers, ceramics and other materials described below. In certain embodiments, the attachment assembly comprises a metal, for example, platinum, rhodium, palladium, rhenium, as well as tungsten, gold, silver, tantalum, and/or alloys thereof, including any of the metals and alloys described below. In a particularly preferred embodiment, the attachment assembly comprises platinum.

The devices described herein also comprise at least one stretch-resistant member. The stretch-resistant member may be exterior and/or interior to the coil, so long as it functions to keep the coil from over-stretching after deployment. Furthermore, the stretch-resistant member may be made up of one or more components, for example a single filament or two filaments that are attached to each other to function as a stretch-resistant member.

The stretch-resistant member(s) may be made of any material, including, but not limited to one or more metals and/or one or more polymers. In certain embodiments, one or more of stretch-resistant member comprise one or more filaments, for example one or more polymeric filaments, such as a suture material. Multiple filamentous elements may be assembled by one or more operations including coiling, twisting, braiding, weaving or knitting of the filamentous elements.

Non-limiting examples of polymers suitable for use in the devices described herein (e.g., attachment assembly, stretch-resistant member and/or coil) include synthetic and natural polymers, such as polyurethanes (including block copolymers with soft segments containing esters, ethers and carbonates), polyethers, polyamides (including nylon polymers and their derivatives), polyimides (including both thermosetting and thermoplastic materials), acrylates (including cyanoacrylates), epoxy adhesive materials (two part or one part epoxy-amine materials), olefins (including polymers and copolymers of ethylene, propylene butadiene, styrene, and thermoplastic olefin elastomers), fluoronated polymers (including polytetrafluoroethylene), polydimethyl siloxane-based polymers, cross-linked polymers, non-cross linked polymers, Rayon, cellulose, cellulose derivatives such nitrocellulose, natural rubbers, polyesters such as lactides, glycolides, trimethylene carbonate, caprolactone polymers and their copolymers, hydroxybutyrate and polyhydroxyvalerate and their copolymers, polyether esters such as polydioxinone, anhydrides such as polymers and copolymers of sebacic acid, hexadecandioic acid and other diacids, or orthoesters may be used.

Thus, polymers used in the devices described herein may include one or more absorbable (biodegradable) polymers and/or one or more non-absorbable polymers. The terms “absorbable” and “biodegradable” are used interchangeable to refer to any agent that, over time, is no longer identifiable at the site of application in the form it was injected, for example having been removed via degradation, metabolism, dissolving or any passive or active removal procedure. Non-limiting examples of absorbable proteins include synthetic and polysaccharide biodegradable hydrogels, collagen, elastin, fibrinogen, fibronectin, vitronectin, laminin and gelatin. Many of these materials are commercially available. Fibrin-containing compositions are commercially available, for example from Baxter. Collagen containing compositions are commercially available, for example from Cohesion Technologies, Inc., Palo Alto, Calif. Fibrinogen-containing compositions are described, for example, in U.S. Pat. Nos. 6,168,788 and 5,290,552. Mixtures, copolymers (both block and random) of these materials are also suitable.

Preferred biodegradable polymers include materials used as dissolvable suture materials, for instance polyglycolic and/or polylactic acids (PGLA) to encourage cell growth in the aneurysm after their introduction. Preferred non-biodegradable polymers include polyethylene teraphthalate (PET or Dacron), polypropylene, polytetraflouroethylene, or Nylon materials. Highly preferred are PET or PGLA.

At least one component of the stretch-resistant member(s) is preferably secured to the attachment assembly. This can be accomplished by any suitable means, including, but not limited to, tying, winding gluing, melting, etc. For example, a stretch-resistant member can be attached to the attachment assembly by extending the stretch-resistant members through the lumen(s) of the attachment assembly and knotting the stretch resistant members on one or both sides of the attachment assembly.

Thus, as shown in FIG. 2, a stretch-resistant member made up of at least one filament 20 can be attached to the attachment assembly 10 by knotting 25, 26 the filament(s) 20 on the distal and proximal sides of the attachment assembly 10. Securing the filament 20 to both proximal and distal sides of the attachment assembly 10 allows the operator to sever each side without affecting the stretch-resistance or coil securement function of the other side. Thus, the implantable device distal to the attachment assembly will resist stretching when the filament is severed proximally to the attachment assembly 10 and/or when the filament extending from the distal end of the attachment assembly to the delivery device is severed, the delivery device (e.g., delivery coil) remains attached to the delivery device.

In addition, the devices described herein also include at least one implantable device, for example, an implantable vaso-occlusive device. The implantable may be made of a variety of materials (e.g., metal, polymer, etc.), including the polymers and metals described above. Although depicted in the Figures as a helically wound coil, it will be appreciated that the drawings are for purposes of illustration only and that other embolic devices may be of a variety of shapes or configuration including, but not limited to, open and/or closed pitch helically wound coils, braids, wires, knits, woven structures, tubes (e.g., perforated or slotted tubes), injection-molded devices and the like. See, e.g., U.S. Pat. No. 6,533,801 and International Patent Publication WO 02/096273.

In a particularly preferred embodiment, the implantable device comprises at least one metal or alloy. Suitable metals and alloys for use in the implantable device, attachment assembly and/or filament(s) include the Platinum Group metals, especially platinum, rhodium, palladium, rhenium, as well as tungsten, gold, silver, tantalum, and alloys of these metals. In one preferred embodiment, the implantable device comprises platinum. The implantable device may also comprise of any of a wide variety of stainless steels if some sacrifice of radio-opacity may be tolerated. Very desirable materials of construction, from a mechanical point of view, are materials that maintain their shape despite being subjected to high stress.

Certain “super-elastic alloys” include nickel/titanium alloys (48-58 atomic % nickel and optionally containing modest amounts of iron); zinc alloys (38-42 weight % zinc); copper/zinc alloys containing 1-10 weight % of silicon, tin, aluminum, or gallium; or nickel/aluminum alloys (36-38 atomic % aluminum) may also be used to make the implantable device, attachment assembly and/or in the filaments of the stretch-resistant devices described herein. Particularly preferred for the implantable device are the alloys described in U.S. Pat. Nos. 3,174,851; 3,351,463; and 3,753,700. Especially preferred is the titanium/nickel alloy known as “nitinol.” These are very sturdy alloys that will tolerate significant flexing without deformation even when used as a very small diameter wire. If a super-elastic alloy such as nitinol is used in any component of the device, the diameter of the wire may be significantly smaller than that used when the relatively more ductile platinum or platinum/tungsten alloy is used as the material of construction. These metals have significant radio-opacity and in their alloys may be tailored to accomplish an appropriate blend of flexibility and stiffness. They are also largely biologically inert.

The implantable device may have a primary and secondary (relaxed configuration). In certain embodiments, the implantable device changes shape upon deployment, for example change from a constrained linear form to a relaxed, three-dimensional (secondary) configuration. See, also, U.S. Pat. No. 6,280,457 and documents cited above for methods of making vaso-occlusive coils having a linear helical shape and/or a different three-dimensional (secondary) configuration.

Thus, it is further within the scope of this invention that the vaso-occlusive device as a whole or elements thereof comprising secondary shapes or structures that differ from the linear coil shapes depicted in the Figures, for examples, spheres, ellipses, spirals, ovals, figure-8 shapes, etc. The devices described herein may be self-forming in that they assume the secondary configuration upon deployment into an aneurysm. Alternatively, the devices may assume their secondary configurations under certain conditions (e.g., change in temperature, application of energy, etc.).

In a preferred embodiment, the implantable device comprises a metal wire wound into a primary helical shape. The implantable device may be, but is not necessarily, subjected to a heating step to set the wire into the primary shape. The diameter of the wire typically making up the coils is often in a range of 0.0005 and 0.050 inches, preferably between about 0.001 and about 0.004 inches in diameter.

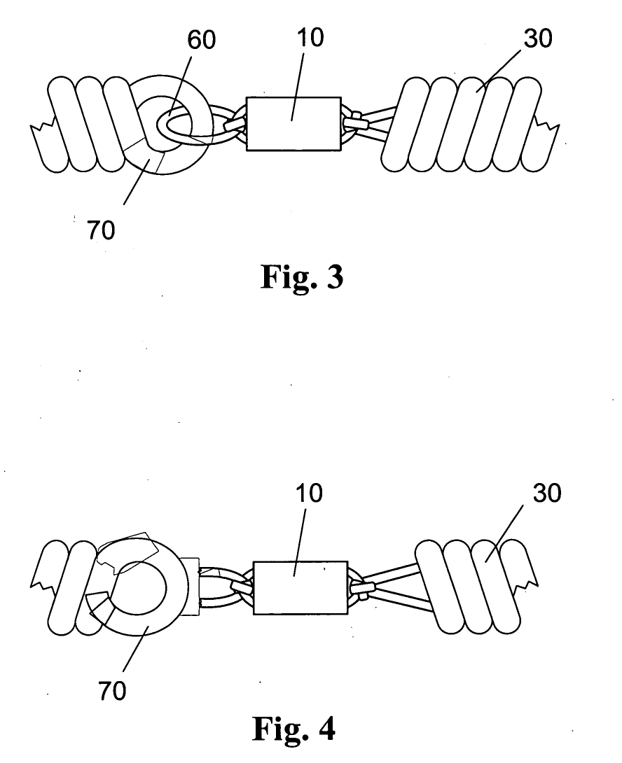

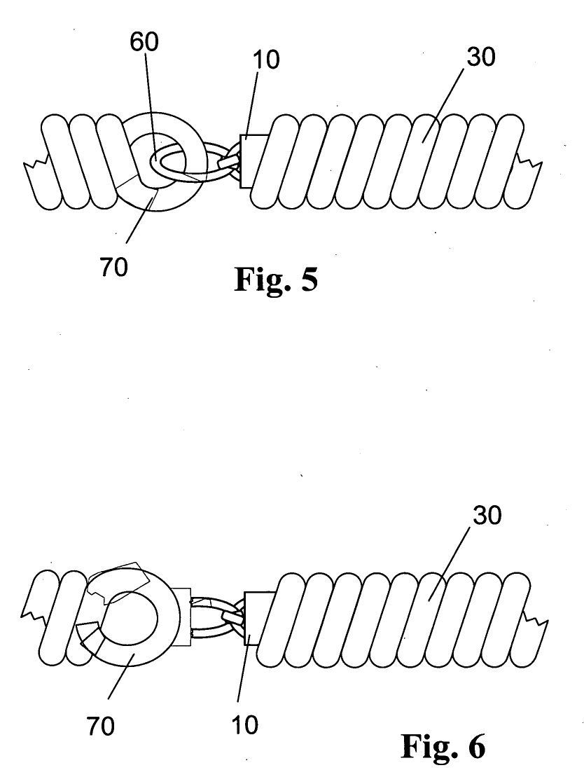

In the devices described herein, the attachment assembly may be placed at any position relative to the proximal end of the implantable device. Thus, the attachment assembly can be proximal to, flush with, extend over the exterior surface, or into the lumen, of the device (e.g., coil). FIGS. 3 and 4 show an exemplary embodiment in which the attachment assembly 10 is proximal to the implantable coil 30. FIGS. 5 and 6 show another exemplary embodiment in which a distal portion of the attachment assembly 10 is within the lumen of the proximal region of the implantable coil 30 and a proximal portion of the attachment assembly 10 extends out from the lumen of the coil 30.

The stretch-resistant devices comprising an attachment assembly described herein are combined with a vaso-occlusive implantable device so as to inhibit unwanted stretching of the vaso-occlusive implantable device after deployment. Thus, it is preferably that the attachment assembly be structured so that it is unable to travel any significant length of the coil (e.g., through the lumen), which would release the tension on the stretch resistant member. Non-limiting examples of how this can be accomplished include sizing the diameter of the attachment assembly to be slightly larger than the diameter of the lumen of the coil so that the attachment assembly cannot travel through the coil. Alternatively, the attachment assembly may be fixed (e.g., gluing, soldering, melting, etc.) to the coil (exterior and/or in lumen) so that it does not move in relation to the coil.

The attachment assembly and/or stretch resistant member(s) is(are) also preferably secured to the implantable device in any fashion, including, but not limited to, melting, by adhesives (e.g., EVA), tying, winding and the like. The stretch-resistant member may be attached to the implantable device at one or more locations, for example, the distal end of the coil. In certain embodiments, the stretch-resistant member is formed from two separate structures. For example, the stretch-resistant member(s) attached to the attachment assembly may form a loop structure that can extend from the distal end of the attachment assembly into the lumen of the implantable device (coil). One or more additional stretch-resistant members within the coil (e.g., secured at or near the distal end of the coil) can then be attached to the stretch-resistant member extending distally from the attachment assembly, for example by tying, looping, melting.

The attachment assembly and/or stretch-resistant member(s) may be combined with the implantable device before or after the implantable device is shaped into a primary and/or secondary configuration. For example, the implantable device may be formed into its primary configuration, and the attachment assembly and/or stretch resistant member secured to the primary configuration as desired. Alternatively, the primary configuration can be shaped into its secondary form and heat treated so that it will return to the secondary form when relaxed (deployed). The attachment assembly and/or stretch-resistant member may then be secured to the implantable device as desired. Whatever combination strategy is employed, the attachment assembly and stretch-resistant member(s) do not substantially affect the shape of the implantable device when the implantable device assumes the relaxed (secondary) configuration.

The devices may also comprise a detachment junction. Suitable detachment junctions will be known to the skilled artisan and include, but are not limited to, electrolytically detachable junctions, mechanically detachable junctions (upon movement or pressure) and/or junctions that are detached the application of heat (thermally detachable), radiation and/or electromagnetic radiation. Methods of connecting a implantable device to a pusher wire having an electrolytically detachable junction are also well known and described for example in U.S. Pat. Nos. 6,620,152; 6,425,893; 5,976,131 5,354,295; and 5,122,136.

The detachment junction may be a separate element added to the device or may be formed from one or more parts of the device. For example, FIGS. 3 and 5 show exemplary embodiments in which a detachment junction 60 is formed by securing the stretch-resistant member extending from the proximal end of the attachment assembly 10 to a distal loop 70 structure on a delivery loop 70. In a preferred embodiment, stretch-resistant vaso-occlusive devices as described herein are conveniently detached from the deployment mechanism (e.g., delivery loop) by the application of thermal and/or electrical energy, which severs the attachment assembly and/or implantable device from the delivery mechanism and releases the device into the selected site. FIGS. 4 and 6 show the assemblies of FIGS. 3 and 5, respectively, after severing of the detachment junction.

Thus, also provided herein are assemblies comprising any of the vaso-occlusive devices described herein in combination with a delivery device. Any delivery device can be employed, including but not limited to, a pusher wire, a delivery device comprising a delivery coil and the like. See, e.g., FIGS. 3-6.

One or more of the components of the devices described herein (e.g., attachment assembly, stretch-resistant member, implantable device) may also comprise additional components, such as co-solvents, plasticizers, radio-opaque materials (e.g., metals such as tantalum, gold or platinum), coalescing solvents, bioactive agents, antimicrobial agents, antithrombogenic agents, antibiotics, pigments, radiopacifiers and/or ion conductors which may be coated using any suitable method or may be incorporated into the element(s) during production.

In addition, lubricious materials (e.g., hydrophilic) materials may be used to coat one or more members of the device to help facilitate delivery. Cyanoacrylate resins (particularly n-butylcyanoacrylate), particular embolization materials such as microparticles of polyvinyl alcohol foam may also be introduced into the intended site after the inventive devices are in place. Furthermore, previously described fibrous braided and woven components (U.S. Pat. No. 5,522,822) may also be included.

One or more bioactive materials may also be included. See, e.g., co-owned U.S. Pat. No. 6,585,754 and WO 02/051460. The term “bioactive” refers to any agent that exhibits effects in vivo, for example a thrombotic agent, an anti-thrombotic agent (e.g., a water-soluble agent that inhibits thrombosis for a limited time period, described above), a therapeutic agent (e.g., chemotherapeutic agent) or the like. Non-limiting examples of bioactive materials include cytokines; extracellular matrix molecules (e.g., collagen); trace metals (e.g., copper); and other molecules that stabilize thrombus formation or inhibit clot lysis (e.g., proteins or functional fragments of proteins, including but not limited to Factor XIII, α₂-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (PDGF), vascular endothelial growth factor (VEGF), transforming growth factor beta (TGF-β) and the like. Cytokines, extracellular matrix molecules and thrombus stabilizing molecules (e.g., Factor XIII, PAI-1, etc.) are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequences of many of these molecules are also available, for example, from the GenBank database. Thus, it is intended that the invention include use of DNA or RNA encoding any of the bioactive molecules. Cells (e.g., fibroblasts, stem cells, etc.) can also be included. Such cells may be genetically modified. Furthermore, it is intended, although not always explicitly stated, that molecules having similar biological activity as wild-type or purified cytokines, extracellular matrix molecules and thrombus-stabilizing proteins (e.g., recombinantly produced or mutants thereof) and nucleic acid encoding these molecules are intended to be used within the spirit and scope of the invention. Further, the amount and concentration of liquid embolic and/or other bioactive materials useful in the practice of the invention can be readily determined by a skilled operator and it will be understood that any combination of materials, concentration or dosage can be used, so long as it is not harmful to the subject.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the absorbable vaso-occlusive device at the distal end, is advanced through the catheter.

Once the selected site has been reached, the vaso-occlusive device is extruded, for example by severing the detachment junction. For example, the vaso-occlusive device can be loaded onto the pusher wire via an electrolytically or thermally cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. Nos. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising
an implantable device having a proximal end and a distal end;
an attachment assembly having proximal and distal ends, the attachment assembly disposed at or near the proximal end of the implantable device;
at least one stretch-resistant member secured to the implantable device and the attachment assembly.

2. The vaso-occlusive device of claim 1, wherein the attachment assembly comprises at least one lumen therein and wherein at least one stretch-resistant member extends through the lumen of the attachment assembly.

3. The vaso-occlusive device of claim 2, wherein the stretch-resistant member comprises at least one filament.

4. The vaso-occlusive device of claim 1, wherein the stretch-resistant member is secured to the proximal or distal end of the attachment assembly.

5. The vaso-occlusive device of claim 1, wherein the stretch-resistant member is secured to the proximal and distal ends of the attachment assembly.

6. The vaso-occlusive device of claim 4, wherein the stretch-resistant member is secured to the attachment assembly by knotting, adhesive or other bonding.

7. The vaso-occlusive device of claim 1, wherein the attachment assembly comprises a metal.

8. The vaso-occlusive device of claim 7, wherein the metal comprises platinum.

9. The vaso-occlusive device of claim 1, wherein the stretch-resistant member comprises a polymer.

10. The vaso-occlusive device of claim 9, wherein the polymer comprises a suture material.

11. The vaso-occlusive device of claim 1, the implantable device defines a lumen and the attachment assembly extends at least partially into the lumen of the implantable device.

12. The vaso-occlusive device of claim 11, wherein the attachment assembly is secured to the implantable device.

13. The vaso-occlusive device of claim 1, wherein the implantable device comprises a wire formed into a helically wound primary shape.

14. The vaso-occlusive device of claim 1, wherein the implantable device has a secondary shape that self-forms upon deployment.

15. The vaso-occlusive device of claim 14, wherein the secondary shape is selected from the group consisting of cloverleaf shaped, helically-shaped, figure-8 shaped, flower-shaped, vortex-shaped, ovoid, randomly shaped, and substantially spherical.

16. The vaso-occlusive device of claim 1, wherein the implantable device comprises a metal.

17. The vaso-occlusive device of claim 16, wherein the metal is selected from the group consisting of platinum, rhodium, gold, tungsten and alloys thereof.

18. The vaso-occlusive device of claim 17, wherein the metal comprises a nickel-titanium alloy.

19. The vaso-occlusive device of claim 1, further comprising a detachment junction.

20. The vaso-occlusive device of claim 19, wherein the detachment junction is electrolytically or thermally detachable.

21. An assembly comprising:
a vaso-occlusive device according to claim 1; and
a delivery device,
wherein the vaso-occlusive device is secured to the delivery device prior to deployment.

22. A method of at least partially occluding an aneurysm, the method comprising the steps of introducing a vaso-occlusive device according to claim 1 into the aneurysm and detaching the implantable device from the detachment junction, thereby deploying the implantable device into the aneurysm.

23. A method of at least partially occluding an aneurysm, the method comprising the steps of introducing a vaso-occlusive assembly according to claim 21 into the aneurysm and detaching the implantable device from the detachment junction, thereby deploying the implantable device into the aneurysm.
